(19) United States
(12) Plant Patent Application Publication
(10) Pub. No.: US 2004/0168237 P1
(43) Pub. Date: Aug. 26, 2004

(54) Betula papyrifera named ' Varen '

(76) Inventors: Dale E. Herman, Fargo, ND (US); Larry J. Chaput, Fargo, ND (US); Zong-Ming Cheng, Knoxville, TN (US); Wenhao Dai, Fargo, ND (US)

Correspondence Address: JONDLE & ASSOCIATES P.C., 9085 EAST MINERAL CIRCLE, SUITE 200, CENTENNIAL, CO, 80112, US

(21) Appl. No.: 10/377,136

(22) Filed: Feb. 26, 2003

(51) Int. Cl. A01H 5/00

(52) U.S. Cl. PLT/216

(57) ABSTRACT

‘Varen’ is a new cultivar of Betula papyrifera birch tree. This new cultivar has exceptional clear white bark and dark green leaves.

### GENUS AND SPECIES

Betula papyrifera

### CULTIVAR DENOMINATION

‘Varen’

### BACKGROUND OF THE INVENTION

The present invention comprises a new and distinct cultivar of birch tree, botanically known as Betula papyrifera, and hereinafter referred to by the cultivar name ‘Varen’. ‘Varen’ is a product of a planned tree selection program which had the objective of creating a new cultivar of birch tree having high bronze birch borer resistance, good birch leafminer resistance and various aesthetic qualities which were originated in a controlled breeding program.

The new cultivar is a selection of Betula papyrifera, and was discovered by applicant among seedlings grown in NDSU Research Aboretum, Absaraka, N.D. from parental trees growing native in the Killdeer Mountains approximately eight miles northwest of Kilideer, N.D. on Aug. 10, 1976. The selection was noticed by virtue of its long term bronze birch borer resistance, exceptionally white bark, marked exfoliation of bark, quality dark green foliage and excellent golden-yellow autumn foliage coloration, upright, oblong-elliptical form becoming somewhat more rounded with maturity.

The first act of asexual reproduction of ‘Varen’ was accomplished when plants were produced in tissue culture by applicant from the initial selection on Mar. 10, 2000 in a controlled environment in Fargo, N.D. (NDSU). Horticultural examination of selected units initiated has demonstrated that the combination of characteristics as herein disclosed for ‘Varen’ are firmly fixed and are retained through successive generations of asexual reproduction.

‘Varen’ has not been observed under all possible environment conditions. The phenotype may vary with variations in environment such as temperature, light intensity, and day length. The following observations, measurements and comparisons describe the plants grown in NDSU Research Arboretum or under greenhouse conditions, which approximate those generally used in commercial practices.

This new birch tree is illustrated by the accompanying photographs which show blooms, buds, and foliage of the plant in full color, the colors shown being as true as can be reasonably obtained by conventional photographic procedures.

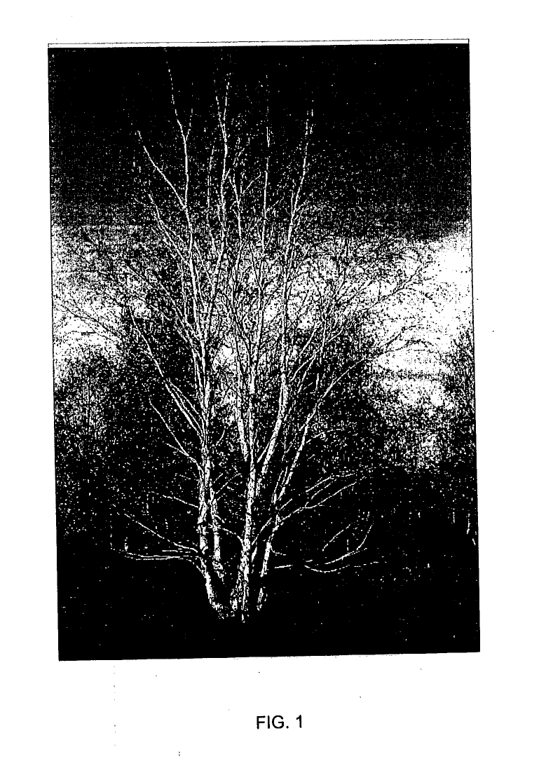

FIG. 1 shows overall tree without foliage;

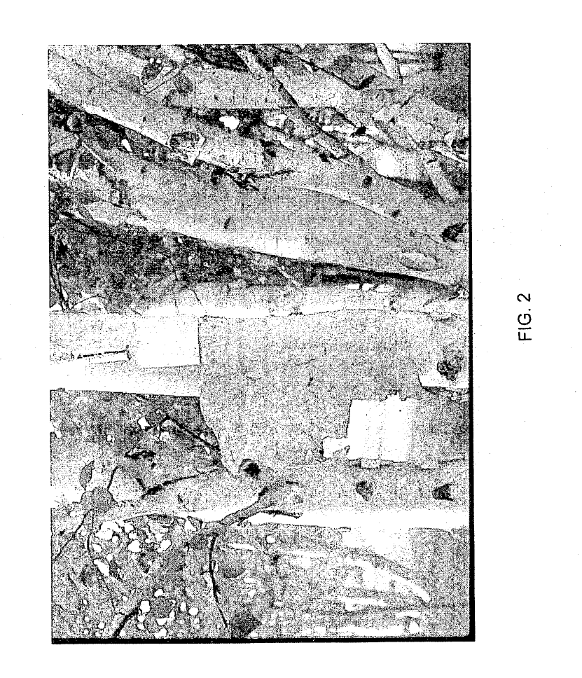

FIG. 2 shows the bark color and exfoliation on the trunks;

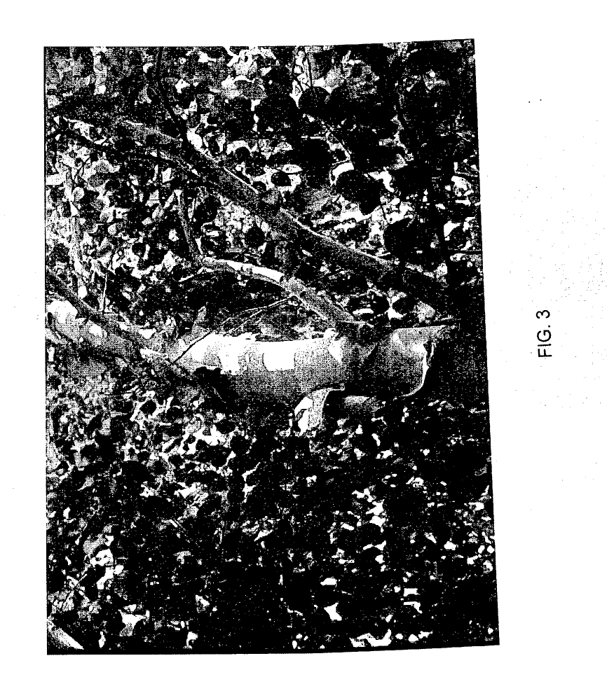

FIG. 3 shows the dense foliage;

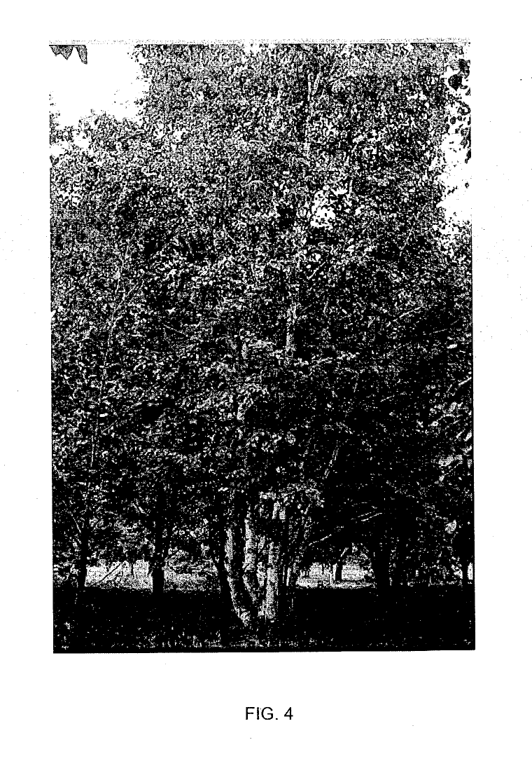

FIG. 4 shows the complete tree in dense foliage; and

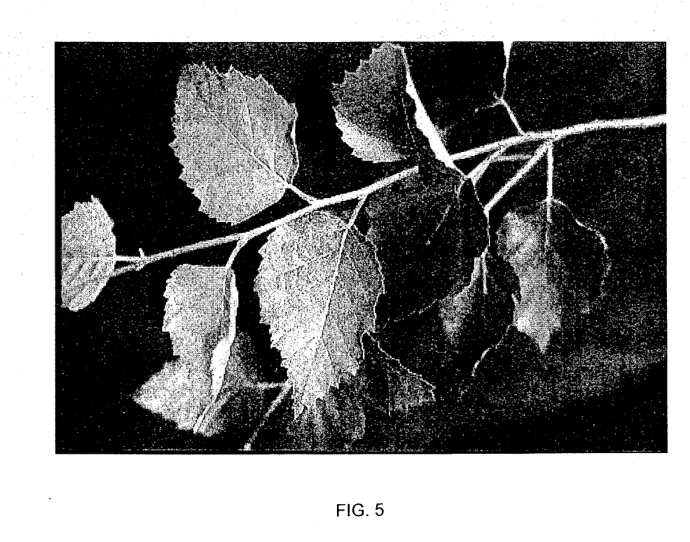

FIG. 5 is a close-up of the leaves.

### DETAILED DESCRIPTION OF THE NEW PLANT

The following traits and characteristics describe the new cultivar.

Classification:

Origin.—Killdeer Mountains, eight miles northwest of Killdeer, N.D.

Species.—Betula papyrifera.

Common names.—Paper Birch.

### TREE

Trunk:

Multiple stem.—Yes, four main trunks forming clump.

Size.—At 25 years of age, average diameter of four trunks, 17.3 cm.

Bark.—Exceptionally clear white bark, average length of exfoliating. sheets, 14.9 cm.

Height.—At 25 years of age, 12.1 m.

Growth habit or form.—Semi-pyramidal to upright, broadly oval.

Growth rate.—48.4 cm/year (under sod conditions in arboretum, no supplementary irrigation or fertilization.

Branches:

Angle of attachment.—Average angle of 26 branches, 49.5 degree.

Spacing.—Variable.

Size.—Crown diameter of tree, 7.3 m at 25 years.

Bark.—Prior to bark changing to white, branches aged 3-7 years, range in color from greyed-orange (RHS 166A) to brown (RHS 200A/B).

Lenticels:

Size.—1-2 mm. on branches less than one inch in diameter.

Color.—Whitish.

Quantity.—Many.

Foliage:

Size of leaf.—Length — Range 7-12 cm, average 9.2 cm Width — Range 5.3-8.8 cm, average 6.5 cm.

Shape of leaf.—Ovate to broadly-ovate Margin.—Doubly-serrate Texture.—Medium.

Pubescence distribution.—Slightly pubescent on both leaf surfaces.

Color.—Upper side, RHS 137A (green) Underside, RHS 147B (yellow-green).

Petiole.—Range from 2.0-3.6 cm, average 2.7 cm Ribs and veins — Average of 8 vein pairs per leaf Thorns and spines — None Buds — Imbricate, pointed, scattered hairs.

### CATKINS

Staminate:

Shape.—Cylindrical, dormant stage fairly rigid, blooming stage, pendulous.

Size.—Dormant stage length 1.4-3.8 cm, average 2.8 cm.

Blooming stage.—6.0-11.5 cm, average 8.6 cm.

Scales:

Shape.—teardrop to diamond-shaped, dormant stage.

Size.—2.0 mm.

Color.—RHS 200D (brown) to RHS 6C/D (yellow).

Pistillate:

Shape.—Cylindrical; blooming stage, not pendulous.

Size.—Blooming stage 1.3-2.2 cm, average 1.6 cm.

Scales:

Shape.—Teardrop to diamond shaped.

Size.—1.1 mm.

Color.—RHS 149A (yellow-green) to RHS 144C (yellow-green).

Timing of appearance, staminate, and pistillate catkins:

Staminate.—Develop during summer.

Pistillate.—Develop May 5-20.

Timing of anthesis.—May 1-15, varies with year.

Fruit: Stobile bearing, many winged nutlets (seeds) which average 6 mm long And 3 mm wide.

Shape.—Cylindrical.

Strobile length.—4.3-5.1 cm, average 4.7 cm.

Strobile width.—0.9-1.2 cm, average 1.1 cm.

Color.—Green, changing to brown in the fall.

### INSECT AND DISEASE RESISTANCE

No evidence of bronze birch borer attack in 25 years of evaluation in large birch collection in NDSU Research Arboretum where borer population was high. Resistance to birch leaf miner has been good to date.

### COMPARISON WITH MOST SIMILAR CULTIVAR

Of the many commercial birch trees known to the applicant, the most similar in comparison to ‘Varen’ are the other introduced Betula papyrifera cultivars such as; Betula papyrifera ‘Oenci’ with broad, pyramidal form, exfoliating white bark, young branches, red-mahogany color.

Betula papyrifera ‘Cenci’ with tight oval, compact growth habit, peeling sheets of white bark.

Betulal papyrifera ‘Renci’ with narrowly pyramidal growth habit, non-exfoliating white bark.

Betula papyrifera ‘Uenci’ with narrow upright growth habit, semi-exfoliating white bark exposing cinnamon under-layer.

‘Chickadee’ Paper Birch with narrow columnar-conical selection, white bark, introduced in Canada. Availability in nursery trade not know.

‘Snowy’ Paper Birch with multi-clonal seedling cultivar originating at Michigan State University. Availability in nursery trade and cultivar name not known. White bark, reputed bronze birch borer resistance.

What is claimed is:

1. A new and distinct Betula papyrifera birch tree as shown and described herein.
